(19) United States
(12) Patent Application Publication
(10) Pub. No.: US 2025/0266164 A1
(43) Pub. Date: Aug. 21, 2025

(54) DIAGNOSIS AID DEVICE USING SMART HEMO-DYNAMIC INDEX AND METHOD THEREOF

(71) Applicants: IUCF-HYU (INDUSTRY-UNIVERSITY COOPERATION FOUNDATION HANYANG UNIVERSITY), Seoul (KR); Seoul National University R&DB Foundation, Seoul (KR); KOREA VETERANS HEALTH SERVICE, Wonju-si, Gangwon-do (KR)

(72) Inventors: Si Mon SONG, Seoul (KR); Don Gwan AN, Seoul (KR); Sang Hyung LEE, Seoul (KR); Doo Sang KIM, Seoul (KR)

(73) Assignees: IUCF-HYU (INDUSTRY-UNIVERSITY COOPERATION FOUNDATION HANYANG UNIVERSITY), Seoul (KR); Seoul National University R&DB Foundation, Seoul (KR); KOREA VETERANS HEALTH SERVICE, Wonju-si, Gangwon-do (KR)

(21) Appl. No.: 18/857,873

(22) PCT Filed: Apr. 20, 2023
(86) PCT No.: PCT/KR2023/005368

§ 371 Date: Oct. 18, 2024

(30) Foreign Application Priority Data
Jan. 21, 2022 (KR) 10-2022-0049283
Apr. 20, 2023 (KR) 10-2023-0051917

(51) Int. Cl. G16H 50/20; G16H 10/60; G16H 30/40

(52) U.S. Cl. CPC G16H 50/20; G16H 10/60; G16H 30/40

(57) ABSTRACT

The present invention relates to a diagnostic aid device and method using smart hemodynamic indices and, more particularly, to a diagnostic aid device and method for aid in medical decision-making, by calculating smart hemodynamic indices, which is a combination of smart indices with hemodynamic indices, and providing diagnosis results according to quantitatively standardized clinical guidelines by using the smart hemodynamic indices.

### TECHNICAL FIELD

The present invention relates to a diagnosis AID DEVICE using a smart hemodynamic index and a method thereof, and more particularly to a technology of aid in medical decision-making by calculating a smart hemodynamic index which is a combination of a smart index and a hemodynamic index, and providing a diagnosis result according to quantitatively normalized clinical guidelines using a smart hemodynamic index.

### BACKGROUND ART

Fractional flow reserve (FFR) is a technology for measuring the intravascular pressure that decreases due to coronary artery stenosis to diagnose the degree of stenosis progression.

An existing method of calculating a hemodynamic index uses only a single pressure index among hemodynamic factors, so there is a gray region in the criteria, and it requires considerable time, cost, and professional manpower for measurement.

That is, a hemodynamic index according to the existing technology is expensive and time-consuming to calculate, so it is wasteful in the judgment of diagnosis and prognosis prediction in a medical diagnosis process.

In addition, the method of calculating a hemodynamic index according to an existing technology has a problem of low reliability because it sometimes does not match the judgment of actual clinicians.

### DISCLOSURE

### Technical Problem

Therefore, the present invention has been made in view of the above problems, and it is one object of the present invention to calculate a smart hemodynamic index which is a combination of a smart index and a hemodynamic index, and provide a diagnosis result according to quantitatively normalized clinical guidelines using the smart hemodynamic index, thereby providing a diagnosis AID DEVICE for supporting medical decision-making and a method thereof.

It is another object of the present invention to provide a hemodynamic index and a smart index, which are innovative diagnostic technologies capable of expressing the analysis process and results, which require a high level of understanding of both hemodynamics and medicine, as quantitatively normalized numbers and capable of suggesting a clinical guideline that matches the numbers, and a smart hemodynamic index obtained by integrating the hemodynamic index and the smart index, thereby innovatively simplifying an existing medical diagnosis process and, at the same time, supporting accurate diagnosis and prognosis prediction through more multifaceted considerations.

It is yet another object of the present invention to calculate a smart index and hemodynamic index, which are indices formulated using information that can be obtained from existing medical devices, and a smart hemodynamic index obtained by integrating the smart index and hemodynamic index. Accordingly, the present invention does not require the purchase of new medical devices, and thus can provide a diagnosis support apparatus, which can be used in all 1st, 2nd, and 3rd medical environments, and a use method thereof.

### Technical Solution

In accordance with an aspect of the present invention, the above and other objects can be accomplished by the provision of a diagnosis AID DEVICE using a smart hemodynamic index, the diagnosis AID DEVICE including: a clinical information collector for collecting clinical information related to a diagnosis subject; a clinical factor calculator for calculating a clinical factor based on the collected clinical information; a hemodynamic factor calculator for calculating a hemodynamic factor based on the collected clinical information; an index calculator for calculating a smart index (SI) using the calculated clinical factor, calculating a hemodynamic index (HDI) using the calculated hemodynamic factor, and calculating a smart hemodynamic index (SHDI) by simultaneously considering the calculated smart index and the calculated hemodynamic index; and a diagnosis result provider for providing a diagnosis result for the diagnosis subject by applying one of the calculated smart index, the calculated hemodynamic index and the calculated smart hemodynamic index to a diagnosis guideline.

The index calculator may calculate a hemodynamic standard factor by normalizing the hemodynamic factor, and calculate the hemodynamic index by assigning a weight to the calculated hemodynamic standard factor.

The index calculator may calculate a clinical standard factor by normalizing the clinical factor, and calculate the smart index by assigning a weight to the calculated clinical standard factor.

The clinical factor calculator may calculate a clinical factor including at least one of diabetes, blood pressure, hyperlipidemia, smoking, hematocrit, cholesterol level, pulse rate, ultrasound image, CT image, MRI image, pain, heart failure, blood concentration in the body, blood inflammation level, myocardial perfusion single photon tomography image, underlying disease, heredity, body weight and age based on disease-specific characteristics in the clinical information.

The diagnosis result provider may provide clinical information, related to the diagnosis subject, to multiple medical professionals, and then, by applying a score, based on one of the indices, to a diagnosis guideline determined based on a diagnosis result, provide a diagnosis result corresponding to the applied score in the diagnosis guideline.

The clinical information collector may collect a medical image including at least one of angiographic images, ultrasound Doppler images and CT images according to clinical information acquisition criteria, and collect data on 3D space and 4D blood flow information as clinical information by applying an artificial intelligence prediction model to the collected medical image.

The hemodynamic factor calculator may calculate a hemodynamic factor including at least one of basic blood flow information factors, velocity factors, flow factors, secondary flow factors, wall stress-based factors, pressure factors and morphological factors based on four-dimensional blood flow velocity information in the clinical information.

The index calculator may calculate a hemodynamic standard factor by assigning a score in a predefined score range according to the calculated hemodynamic factor, classify a disease into one among a plurality of diseases according to the calculated hemodynamic standard factor, and calculate a hemodynamic index by assigning a weight, calculated according to a degree of disease, to the calculated hemodynamic standard factor.

The index calculator may classify the calculated hemodynamic factor into multiple cases, calculate a threshold value for each disease based on an average value of each of the classified case, and calculate a hemodynamic standard factor by assigning a score in the predefined score range based on the calculated threshold value.

The index calculator may calculate a clinical standard factor by assigning a score in a predefined score range according to the calculated clinical factor, classify a disease into one of a plurality of diseases according to the calculated clinical standard factor, and calculate a smart index by assigning a weight, calculated according to a degree of disease, to the calculated clinical standard factor.

In accordance with another aspect of the present invention, provided is a diagnosis AID METHOD using a smart hemodynamic index, the diagnosis AID METHOD including: collecting, by a clinical information collector, clinical information related to a diagnosis subject; calculating, by a clinical factor calculator, a clinical factor based on the collected clinical information; calculating, by a hemodynamic factor calculator, a hemodynamic factor based on the collected clinical information; calculating, by an index calculator, a smart index (SI) using the calculated clinical factor, calculating a hemodynamic index (HDI) using the calculated hemodynamic factor, and calculating a smart hemodynamic index (SHDI) configured by considering the calculated smart index and the calculated hemodynamic index; and providing, by a diagnosis result provider, a diagnosis result for the diagnosis subject by applying one of the calculated smart index, the calculated hemodynamic index and the calculated smart hemodynamic index to a diagnosis guideline.

The calculating by the index calculator may include: calculating a hemodynamic standard factor by normalizing the hemodynamic factor, and calculating the hemodynamic index by assigning a weight to the calculated hemodynamic standard factor, calculating a clinical standard factor by normalizing the clinical factor, and calculating the smart index by assigning a weight to the calculated clinical standard factor; and calculating a clinical standard factor by normalizing the clinical factor, and calculating the smart index by assigning a weight to the calculated clinical standard factor.

The providing by the diagnosis result provider may include: providing clinical information, related to the diagnosis subject, to multiple medical professionals, and then, by applying a score, based on one of the indices, to a diagnosis guideline determined based on a diagnosis result, providing a diagnosis result corresponding to the applied score in the diagnosis guideline.

The collecting by the clinical information collector may include: collecting a medical image including at least one of angiographic images, ultrasound Doppler images and CT images according to clinical information acquisition criteria, and collecting data on 3D space and 4D blood flow information as clinical information by applying an artificial intelligence prediction model to the collected medical image.

The calculating by the hemodynamic factor calculator may include: calculating a hemodynamic factor including at least one of basic blood flow information factors, velocity factors, flow factors, secondary flow factors, wall stress-based factors, pressure factors and morphological factors based on four-dimensional blood flow velocity information in the clinical information, and the calculating by the index calculator may include: calculating a hemodynamic standard factor by assigning a score in a predefined score range according to the calculated hemodynamic factor, classifying a disease into one among a plurality of diseases according to the calculated hemodynamic standard factor, and calculating a hemodynamic index by assigning a weight, calculated according to a degree of disease, to the calculated hemodynamic standard factor.

The calculating by the hemodynamic factor calculator may include: calculating a hemodynamic factor including at least one of basic blood flow information factors, velocity factors, flow factors, secondary flow factors, wall stress-based factors, pressure factors and morphological factors based on four-dimensional blood flow velocity information in the clinical information.

The calculating by the index calculator may include: calculating a hemodynamic standard factor by assigning a score in a predefined score range according to the calculated hemodynamic factor, classifying a disease into one among a plurality of diseases according to the calculated hemodynamic standard factor, and calculating a hemodynamic index by assigning a weight, calculated according to a degree of disease, to the calculated hemodynamic standard factor.

### Advantageous Effects

The present invention can calculate a smart hemodynamic index which is a combination of a smart index and a hemodynamic index, and provide a diagnosis result according to quantitatively normalized clinical guidelines using the smart hemodynamic index, thereby being capable of providing a diagnosis AID DEVICE for supporting medical decision-making and a method thereof.

The present invention can provide a hemodynamic index and a smart index, which are innovative diagnostic technologies capable of expressing the analysis process and results, which require a high level of understanding of both hemodynamics and medicine, as quantitatively normalized numbers and capable of suggesting a clinical guideline that matches the numbers, and a smart hemodynamic index obtained by integrating the hemodynamic index and the smart index, thereby being capable of innovatively simplifying an existing medical diagnosis process and, at the same time, supporting accurate diagnosis and prognosis prediction through more multifaceted considerations.

The present invention can calculate a smart index and hemodynamic index, which are indices formulated using information that can be obtained from existing medical devices, and a smart hemodynamic index obtained by integrating the smart index and hemodynamic index. Accordingly, the present invention does not require the purchase of new medical devices, and thus can provide a diagnosis support apparatus, which can be used in all 1st, 2nd, and 3rd medical environments, and a use method thereof.

### DESCRIPTION OF DRAWINGS

FIG. 1 is a drawing for explaining a diagnosis AID DEVICE using a smart hemodynamic index according to an embodiment of the present invention.

FIG. 2 is a drawing for explaining the method of calculating a smart hemodynamic index according to an embodiment of the present invention.

FIG. 3 illustrates a method of improving a diagnosis process by applying the smart hemodynamic index according to an embodiment of the present invention.

FIG. 4 illustrates a hemodynamic factor according to an embodiment of the present invention.

FIG. 5 illustrates a configuration for normalizing a hemodynamic factor according to an embodiment of the present invention.

FIG. 6 is a drawing for explaining a normalized clinical guideline according to an embodiment of the present invention.

FIGS. 7a and 7b illustrate a method of calculating the weight of a hemodynamic standard factor according to an embodiment of the present invention.

FIG. 8 is a drawing for explaining a method of providing a current patient status diagnosis result according to one of a smart index, a hemodynamic index and a smart hemodynamic index according to an embodiment of the present invention.

FIG. 9 is a drawing for explaining a diagnosis AID METHOD using a smart hemodynamic index according to an embodiment of the present invention.

### BEST MODE

Hereinafter, the embodiments of the present disclosure will be described in detail with reference to the drawings.

However, it should be understood that the present disclosure is not limited to the embodiments according to the concept of the present disclosure, but includes changes, equivalents, or alternatives falling within the spirit and scope of the present disclosure.

In the following description of the present disclosure, detailed description of known functions and configurations incorporated herein will be omitted when it may make the subject matter of the present disclosure unclear.

In addition, the terms used in the specification are defined in consideration of functions used in the present disclosure, and can be changed according to the intent or conventionally used methods of clients, operators, and users. Accordingly, definitions of the terms should be understood on the basis of the entire description of the present specification.

In description of the drawings, like reference numerals may be used for similar elements.

Singular expressions encompass plural expressions unless clearly specified otherwise in context.

In this specification, the expressions “A or B” and “at least one of A and B” may include all possible combinations of the items listed together.

Expressions “first” and “second” can be used to qualify the components, regardless of order or importance, and are used to distinguish one component from another, and do not limit the components.

It will be understood that when an element (e.g., first) is referred to as being “connected to” or “coupled to” another element (e.g., second), the first element may be directly connected to the second element or may be connected to the second element via an intervening element (e.g., third).

As used herein, “configured to” may be used interchangeably with, for example, “suitable for”, “ability to”, “changed to”, “made to”, “capable of”, or “designed to” in terms of hardware or software.

In some situations, the expression “device configured to” may mean that the device “may do ˜” with other devices or components.

For example, the expression “processor configured to perform A, B, and C” may mean that a general-purpose processor (e.g., CPU or application processor) performs the corresponding operations by executing a dedicated processor (e.g., an embedded processor) or one or more software programs stored in the memory device.

In addition, the expression “or” means “inclusive or” rather than “exclusive or”.

That is, unless mentioned otherwise or clearly inferred from context, the expression “x uses a or b” means any one of natural inclusive permutations.

In the above-described specific embodiments, elements included in the invention are expressed in singular or plural in accordance with the specific embodiments shown.

However, the singular or plural representations are appropriately selected for the situation presented for convenience of explanation, and the above-described embodiments are not limited to the singular or plural constituent elements. In addition, elements may be composed of the singular number, even when the elements are expressed in the plural number, and an element may be composed of the plural number, even when the element is expressed in the singular number.

In addition, the present invention has been described with reference to exemplary embodiments, but it should be understood that various modifications may be made without departing from the scope of the present disclosure.

Therefore, the scope of the present invention should not be limited by the embodiments, but should be determined by the following claims and equivalents to the following claims.

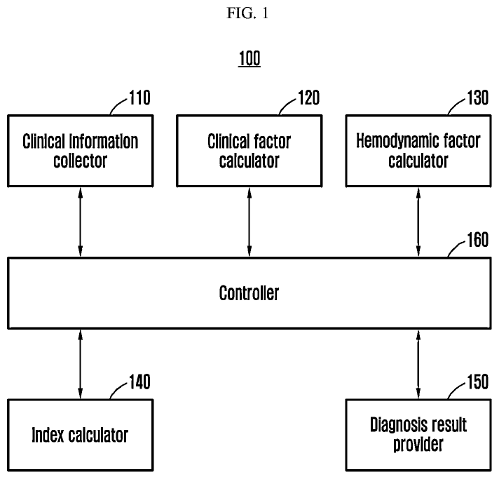

FIG. 1 is a drawing for explaining a diagnosis AID DEVICE using a smart hemodynamic index according to an embodiment of the present invention.

FIG. 1 illustrates components of the diagnosis AID DEVICE using a smart hemodynamic index according to an embodiment of the present invention.

Referring to FIG. 1, the diagnosis AID DEVICE using a smart hemodynamic index 100 according to an embodiment of the present invention includes a clinical information collector 110, a clinical factor calculator 120, a hemodynamic factor calculator 130, an index calculator 140 and a diagnosis result provider 150, wherein a controller 160 controls the functional operations of the above-described components.

In accordance with an embodiment of the present invention, the clinical information collector 110 collects clinical information related to a diagnosis subject.

The diagnosis subject is a patient diagnosed with a disease related to hemodynamics and medicine, and, as a representative disease, there is carotid artery disease. As an example of carotid artery disease, there is carotid artery stenosis.

In accordance with an embodiment of the present invention, the clinical information collector 110 may collect a medical image including at least one of angiographic images, ultrasound Doppler images, and CT images according to clinical information acquisition criteria.

For example, the clinical information collector 110 may collect data on 3D space and 4D blood flow information as clinical information by applying an artificial intelligence prediction model to the collected medical image.

The clinical information includes patient's 4D blood flow velocity information. The blood flow velocity information may mean velocity vector 3 components for 3D space and 1 cardiac cycle time information obtained using methods such as 4D-flow-MRI and computational fluid dynamics (CFD), and wall location information where blood and blood vessels come into contact.

The data format of clinical information is basically entered as a comma-separated value (CSV) format file, but in some cases, it may be any data format that can record and read the velocity vector 3 components (u, v, w) according to the coordinate system (x, y, z, t) such as txt, mat, xlsx, etc. without particular limitation.

In accordance with an embodiment of the present invention, the clinical factor calculator 120 calculates clinical factors based on disease-specific characteristics in clinical information.

For example, the clinical factors may be referred to as clinical parameters, and may include may include at least one of diabetes, blood pressure, hyperlipidemia, smoking, hematocrit, cholesterol level, pulse rate, ultrasound image, CT image, MRI image, pain, heart failure, blood concentration in the body, blood inflammation level, myocardial perfusion single photon tomography image, underlying disease, heredity, body weight and age.

For example, the clinical factor calculator 120 may calculate clinical factors including at least one diabetes, blood pressure, hyperlipidemia, smoking, hematocrit, cholesterol level, pulse rate, ultrasound image, CT image, MRI image, pain, heart failure, blood concentration in the body, blood inflammation level, myocardial perfusion single photon tomography image, underlying disease, heredity, body weight and age based on disease-specific characteristics in clinical information.

In accordance with an embodiment of the present invention, the hemodynamic factor calculator 130 calculates a hemodynamic factor based on four-dimensional blood flow velocity information in clinical information.

For example, a hemodynamic factor may also be referred to as a hemodynamic parameter.

The hemodynamic factor may be calculated based on dimensionless four-dimensional blood flow velocity information, and may include at least one of basic blood flow information factors, velocity factors, flow factors, secondary flow factors, wall stress-based factors, pressure factors and morphological factors.

For example, the hemodynamic factor calculator 130 may calculate a hemodynamic factor including at least one of basic blood flow information factors, velocity factors, flow factors, secondary flow factors, wall stress-based factors, pressure factors and morphological factors based on dimensionless four-dimensional blood flow velocity information.

In accordance with an embodiment of the present invention, the hemodynamic factor calculator 130 may calculate hemodynamic factors from the blood flow velocity information. The calculated hemodynamic factors may be summarized as in Table 1 below.

TABLE 1

| Parameter | |
|---|---|
| Classification | Parameter items |
| Basic information | Blood density, viscosity, heart rate, etc. |
| Velocity | (In the common carotid artery/internal carotid |
| | artery/external carotid artery) average velocity, |
| | peak systolic velocity (PSV), end diastolic |
| | velocity (EDV), etc. |
| Flowrate | (Internal carotid artery vs external carotid |
| | artery) flow rate ratio, flow rate change rate, etc. |
| Secondary flow | Volumes of recirculation zone and flow stasis, |
| | vorticity, helicity, etc. |
| Wall stress | Wall shear stress (WSS), oscillatory shear index |
| | (OSI), relative residence time (RRT), endothelial |
| | cell activation index (ECAP), etc. |
| Pressure | Pressure drop, etc. |
| Morphology | (Internal carotid artery vs external carotid |
| | artery) branch angle, change rate in cross section |
| | of internal carotid artery (ICA) bulb, degree of |
| | stenosis, etc. |

In accordance with an embodiment of the present invention, the hemodynamic factor calculator 130 may mathematically calculate other parameters, except for basic blood flow information and morphological parameters, from the velocity vector.

In this process, the parameters may be the calculated values shown in Table 1, or values obtained after performing a dimensionless process for the calculated values.

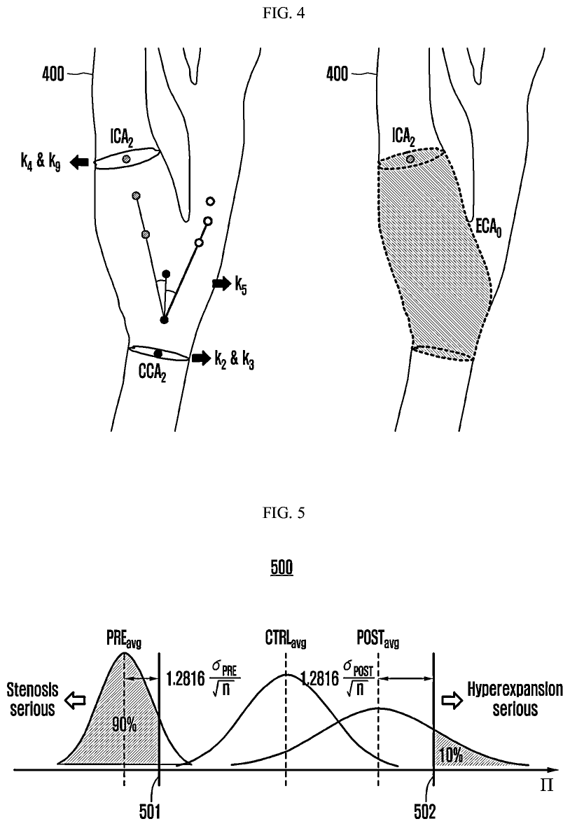

Using FIG. 4, a supplementary explanation is given about for the hemodynamic factors or parameters.

For example, the index calculator 140 may calculate a hemodynamic standard factor by normalizing hemodynamic factors, and may calculate a hemodynamic index by weighting the calculated hemodynamic standard factor.

In accordance with an embodiment of the present invention, the index calculator 140 may calculate a clinical standard factor obtained by normalizing clinical factors, and may calculate a smart index by weighting the calculated clinical standard factor.

In accordance with an embodiment of the present invention, the index calculator 140 may calculate a smart index using the calculated clinical factors, calculate a hemodynamic index using the calculated hemodynamic factors, and may calculate a smart hemodynamic index by simultaneously considering the smart index and the hemodynamic index.

The smart hemodynamic index is an index that is composed by simultaneously considering the smart index and hemodynamic index.

Accordingly, the smart hemodynamic index may have different weights for factors used in the existing smart index and hemodynamic index, or different formulas for score distribution (standard factor).

For example, the index calculator 140 may calculate a hemodynamic standard factor by assigning a score, within a predefined score range, according to hemodynamic factors, classify a disease into one of multiple diseases according to the calculated hemodynamic standard factor, and calculate a hemodynamic index by assigning a weight, calculated according to a simple linear regression analysis according to the degree of the disease, to the calculated hemodynamic standard factor.

In accordance with an embodiment of the present invention, the index calculator 140 may calculate a hemodynamic standard factor by dividing the calculated hemodynamic factor into multiple cases, calculating a threshold value for each disease based on an average value of each of the divided cases, and assigning a score, within a predefined score range, based on the calculated threshold value.

For example, the index calculator 140 may assign a score to each hemodynamic parameter in a predefined score range (0 to 10 points) based on parameter values.

Using FIG. 5, the hemodynamic standard factor related to the hemodynamic factors is supplementally explained.

The index calculator 140 according to an embodiment of the present invention may calculate a clinical standard factor by assigning a score, in a predefined score range, according to the calculated clinical factors, classify a disease into one of a plurality of diseases according to the calculated clinical standard factor, and calculate a smart index by assigning a weight, calculated according to the simple linear regression analysis according to the degree of the disease, to the calculated clinical standard factor.

For example, the index calculator 140 may calculate a smart index using Equation 1 below, calculate a hemodynamic index using Equation 2 below, and calculate a smart hemodynamic index using Equation 3.

$$SI = \sum {w}_{i} \cdot {s}_{i} {s}_{i} = \frac{{p}_{i} - {p}_{i , min}}{{p}_{i , max}} \qquad [ Equation 1 ]$$

In Equation 1, SI may represent a smart index, pᵢ may represent a clinical factor value, sᵢ may represent a clinical standard factor, and wᵢ may represent a weight.

$$HDI = \sum {w}_{i} \cdot {h}_{i} {h}_{i} = \frac{{p}_{i} - {p}_{i , min}}{{p}_{i , max}} \qquad [ Equation 2 ]$$

In Equation 2, HDI may represent a hemodynamic index, pᵢ may represent a hemodynamic factor value, hᵢ may represent hemodynamic standard factor, and wᵢ may represent a weight.

$$SHDI = f ( SI , HDI ) \qquad [ Equation 3 ]$$

In Equation 3, SHDI may represent a smart hemodynamic index, f may represent an integrated function, SI may represent a smart index, and HDI may represent a hemodynamic index.

Hemodynamic indices may be designed by normalizing hemodynamic factors such as existing Fractional Flow Reserve (FFR), Coronary Flow Reserve (CFR), Index of Microcirculatory Resistance (IMR), Wall Shear Stress (WSS), and Oscillatory Shear Index (OSI) to reflect the characteristics of each cardiovascular region and disease, and by quantitatively formulating them through a weighted-sum model and population study.

Hemodynamic indices can provide clear clinical guidelines for diagnosis and prognosis prediction of patients based on hemodynamic indices through a large number of clinical studies.

The smart index is designed as a single integrated index that quantitatively evaluates clinical information such as CT angiography currently used by clinicians for cerebrovascular diagnosis, other diseases such as diabetes or hypertension, and blood component clinical information such as hematocrit volume or cholesterol levels according to the correlation between cardiovascular diseases and perform risk stratification, and is designed to have a high degree of agreement with existing medical decisions, so that it can serve as a quantitative guideline.

The parameters in Table 1 may be configured to assign physical meanings using non-dimensionless.

Dimensionless mathematical equations may be exemplified as in Equations 4 and Equation 5.

$${h}_{1} = \frac{\frac{1}{T} {\int }_{0}^{T} \vert \vec{WSS} \vert dt}{\frac{1}{2} ρ {\vec{V}}^{2}} = \frac{Wall shear stress}{Kinetic energy} \qquad [ Equation 4 ]$$

In Equation 4, h₁ may represent a hemodynamic score based on a scoring equation defined for each parameter of dimensionless hemodynamic parameters.

$${h}_{2} = \frac{{\int }_{}^{} \vec{V} \cdot d \vec{A} {\vert}_{ICA}}{{\int }_{}^{} \vec{V} \cdot d \vec{A} {\vert}_{CCA}} = \frac{( Internal carotid artery flow )}{( Common carotid artery flow )} \qquad [ Equation 5 ]$$

In Equation 5, h₂ may represent a hemodynamics score based on a scoring equation defined for each parameter of dimensionless hemodynamic parameters.

A smart hemodynamic index is a single index that simultaneously considers a hemodynamic index and a smart index.

A method of configuring a smart index and a smart hemodynamic index may be similar to a hemodynamic index.

In accordance with an embodiment of the present invention, the diagnosis result provider 150 may apply one of the calculated hemodynamic index and the calculated smart hemodynamic index to the diagnosis guidelines to provide a diagnosis result for a diagnosis subject.

For example, the diagnosis result provider may provide clinical information related to the diagnosis subject to multiple medical professionals, and then apply a score, based on one index, to the diagnosis guidelines determined based on the diagnosed results, and provide a diagnosis result corresponding to the applied score in the diagnosis guidelines.

For example, the diagnosis result provided by the diagnosis result provider 150 may be utilized as diagnostic support information for diagnosing a patient's disease, illness, etc.

Unlike the existing FFR technology, the smart hemodynamic index of the present invention may be calculated by considering a large number of hemodynamic factors and clinical factors in a complex manner, so that there may be no or small gray area in the quantitative judgment criteria, and thus it may be relatively clear. Accordingly, the smart hemodynamic index is likely to be consistent with actual clinical judgment.

In addition, an existing diagnosis technology based on hemodynamic factors requires a high level of expertise in fluid dynamics, and when used in clinical practice, it has the disadvantage of having to go through a comprehensive diagnosis/judgment process for both clinical and epidemiological aspects, whereas the smart hemodynamic index according to the present invention provides a simple and clear index, allowing for quick decision-making during diagnosis.

In addition, the smart hemodynamic index of the present invention is formulated using information that can be obtained from existing medical devices and hemodynamic analysis methods, so it does not require the purchase of new medical devices and may be used in all 1st, 2nd, and 3rd medical environments.

In addition, due to aging, the incidence of cardiovascular diseases continues to increase and, accordingly, the medical expenses burden increases, so it is predicted that the utilization of the smart hemodynamic index will be very high.

However, effects of the present invention are not limited to the effects described above, and may be variously expanded within a scope that does not depart from the spirit and scope of the present invention.

Accordingly, the present invention may calculate a smart hemodynamic index which is a combination of a smart index and a hemodynamic index, and may provide a diagnosis result according to quantitatively normalized clinical guidelines using the smart hemodynamic index, thereby providing a diagnosis AID DEVICE for supporting medical decision-making and a method thereof.

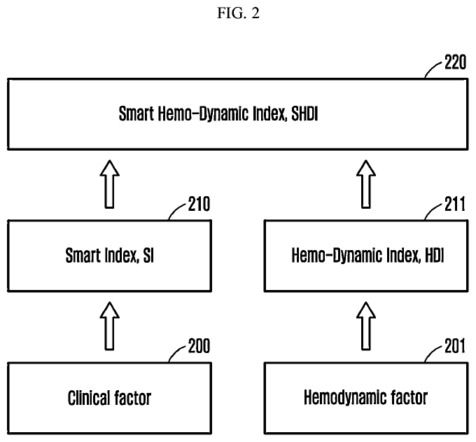

FIG. 2 is a drawing for explaining the method of calculating a smart hemodynamic index according to an embodiment of the present invention.

FIG. 2 illustrates a procedure of calculating a smart hemodynamic index by the method of calculating a smart hemodynamic index according to an embodiment of the present invention.

Referring to FIG. 2, the method of calculating a smart hemodynamic index according to an embodiment of the present invention includes a procedure of calculating a smart index 210 and hemodynamic index 211 using a clinical factor 200 and a hemodynamic factor 201, and calculating a smart hemodynamic index 220 while simultaneously considering the calculated indices.

The method of calculating a smart hemodynamic index according to an embodiment of the present invention collects clinical information obtained through computer simulation or real-time measurement technology (4-D Flow MRI, 3D ultrasound, etc.) of a patient's 4D (time+3D space) pulsatile vascular velocity field using angiographic images and ultrasound Doppler, and through artificial intelligence prediction that learns these.

The method of calculating a smart hemodynamic index according to an embodiment of the present invention calculates various hemodynamic factors (FFR, CFR, IMR, WSS, OSI, etc.) and clinical factors (vascular stenosis rate, concomitant disease, blood flow components, etc.) quantified using clinical information and a velocity field.

The method of calculating a smart hemodynamic index calculates the smart index 210 and the hemodynamic index 211 through normalization and formulating to suit the characteristics of each cardiovascular disease, and calculates the smart hemodynamic index 220 by simultaneously considering the smart index 210 and the hemodynamic index 211.

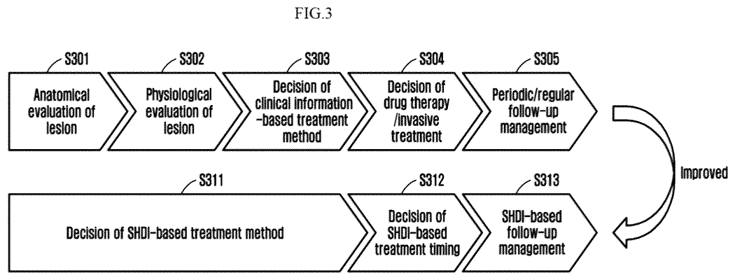

FIG. 3 illustrates a method of improving a diagnosis process by applying the smart hemodynamic index according to an embodiment of the present invention.

FIG. 3 illustrates improvement effects in a diagnosis process by applying the smart hemodynamic index according to an embodiment of the present invention.

Referring to FIG. 3, steps S301 to S305 show a diagnostic procedure according to an existing process, and steps S311 to S313 show an improved diagnosis process according to the present invention.

According to the existing process, an anatomical evaluation of a lesion is performed in step S301, a physiological evaluation of the lesion is performed in step S302, a decision on treatment method based on clinical information is made in step S303, a decision on drug treatment and invasive treatment is made in step S304, and periodic or regular follow-up management is performed in step S305.

A diagnosis AID METHOD according to an embodiment of the present invention includes step 311 in which steps S301 to S303 according to existing methods in the diagnosis and management processes based on a smart hemodynamic index (SHDI) are integrated and simplified.

The diagnosis AID METHOD according to an embodiment of the present invention determines an SHDI-based treatment method in step S311, determines an SHDI-based treatment timing in step S312, and performs SHDI-based follow-up management in step S313.

Step S301 takes about 10 hours for expert-based vascular shape extraction, step S302 takes about 5 hours for computer simulation-based FFR presentation, and steps S303 and S304 are processes for conducting clinical treatment after pre-knowledge of medical data and take a long time.

In addition, step S305 may correspond to inefficient periodic examination and prognostic management for reoperation.

Meanwhile, step S311 reduces human and time consumption as AI-based automatic vascular shape extraction takes about 5 minutes, and improves fidelity and information utilization as AI-based SHDI presentation.

Step S312 enhances clinical convenience and on-site response capability by accessing XR-based real-time medical data and performing procedures.

Step S313 is a step for SHDI-based prognostic prediction and customized management plan establishment, thereby increasing prognostic management efficiency and preventing emergencies in advance.

That is, the diagnosis AID METHOD according to an embodiment of the present invention improves the convenience of diagnosis and treatment subjects based on SHDI, while also supporting a practitioner in making quicker judgments and treatments.

FIG. 4 illustrates a hemodynamic factor according to an embodiment of the present invention.

FIG. 4 illustrates the carotid artery in relation to the hemodynamic factor according to an embodiment of the present invention.

Referring to FIG. 4, carotid artery 400 may be model data analyzed based on velocity information obtained using data obtained by 4D MRI.

Hemodynamic parameters related to the carotid artery 400 may be organized as shown in the following table.

In the carotid artery 400, hemodynamic parameters, Internal Carotid Artery (ICA), External Carotid Artery (ECA) and Common Carotid Artery (CCA) are shown.

The hemodynamic parameters can be summarized as shown in Table 2 below. The hemodynamic parameters are represented as k, and concerning the dimensions, MLT (M: mass, L: length, T: time) are shown.

Dimensions can dimensionally represent information about mass, length, and time.

TABLE 2

| | | Dimensions | | |
|---|---|---|---|---|
| kₐ | Parameters | M | L | T |
| k₁ | Blood density | 1 | −3 | 0 |
| k₂ | Hydraulic diameter of CCA | 0 | 1 | 0 |
| k₃ | Time-averaged bulk velocity of CCA | 0 | 1 | −1 |
| k₄ | Cross-section area of ICA bulb | 0 | 2 | 0 |
| k₅ | Angular ratio of ICA to ECA | 0 | 2 | 0 |
| k₆ | Dynamic viscosity of blood | 1 | −1 | −1 |
| k₇ | Heart rate | 0 | 0 | −1 |
| k₈ | Dynamic range of flow in CCA | 0 | 3 | −1 |
| k₉ | Time-averaged flow of ICA | 0 | 3 | −1 |
| k₁₀ | Time-averaged recirculation amount | 0 | 3 | 0 |
| k₁₁ | Time-averaged stagnation volume | 0 | 3 | 0 |
| k₁₂ | Volume-averaged stagnation of ICA bulb | 0 | 0 | 0 |
| k₁₃ | Time average of WSS | 1 | −1 | −2 |
| k₁₄ | OSI | 0 | 0 | 0 |
| k₁₅ | Area of high shear region | 0 | 2 | 0 |
| k₁₆ | Area of stenosis-prone part | 0 | 2 | 0 |
| k₁₇ | Pressure decrease from CCA₂ to ICA₂ | 1 | −1 | −2 |

The hemodynamic parameter may be mathematically calculated based on velocity information in the carotid artery 400.

$${Π}_{i} = {k}_{i} \cdot {k}_{1}^{a} \cdot {k}_{2}^{b} \cdot {k}_{3}^{c} \qquad [ Equation 6 ]$$

In Equation 6, Π may represent a hemodynamic factor that has a physical meaning as a dimensionless result, and k may represent a value before the dimensionless of the hemodynamic factor. These may be summarized in Table 3 below.

TABLE 3

| Physical | | | | | | | | | | | | | | | | | | | | |
|---|---|---|---|---|---|---|---|---|---|---|---|---|---|---|---|---|---|---|---|---|
| meaning | k₁ | k₂ | k₃ | k₄ | k [symbol] | k [symbol] | k [symbol] | k [symbol] | k [symbol] | k [symbol] | k [symbol] | k [symbol] | k [symbol] | k [symbol] | k [symbol] | k [symbol] | k [symbol] | k [symbol] | k [symbol] | k [symbol] |
| ICA bulb/CCA | 0 | −2 | 0 | 1 | — | — | — | — | — | — | — | — | — | — | — | — | — | — | — | — |
| cross-section | | | | | | | | | | | | | | | | | | | | |
| ratio | | | | | | | | | | | | | | | | | | | | |
| ICA/ECA | 0 | 0 | 0 | — | 1 | — | — | — | — | — | — | — | — | — | — | — | — | — | — | — |
| angle ratio | | | | | | | | | | | | | | | | | | | | |
| Reynolds | 1 | 1 | 1 | — | — | 1 | — | — | — | — | — | — | — | — | — | — | — | — | — | — |
| number, Re | | | | | | | | | | | | | | | | | | | | |
| Strouhal | 0 | 1 | −1 | — | — | — | 1 | — | — | — | — | — | — | — | — | — | — | — | — | — |
| number, St | | | | | | | | | | | | | | | | | | | | |
| (Qₘₐₓ-Qₘᵢₙ)/ | 0 | −2 | −1 | — | — | — | — | 1 | — | — | — | — | — | — | — | — | — | — | — | — |
| Qavg | | | | | | | | | | | | | | | | | | | | |
| Time-average | 0 | −2 | −1 | — | — | — | — | — | 1 | — | — | — | — | — | — | — | — | — | — | — |
| Q [symbol] /Q [symbol] | | | | | | | | | | | | | | | | | | | | |
| Recirculation | 0 | −3 | 0 | — | — | — | — | — | — | 1 | — | — | — | — | — | — | — | — | — | — |
| area size | | | | | | | | | | | | | | | | | | | | |
| compared | | | | | | | | | | | | | | | | | | | | |
| to CCA | | | | | | | | | | | | | | | | | | | | |
| Stagnation | 0 | −3 | 0 | — | — | — | — | — | — | — | 1 | — | — | — | — | — | — | — | — | — |
| area size | | | | | | | | | | | | | | | | | | | | |
| compared | | | | | | | | | | | | | | | | | | | | |
| to CCA | | | | | | | | | | | | | | | | | | | | |
| Stagnation | 0 | 0 | 0 | — | — | — | — | — | — | — | — | 1 | — | — | — | — | — | — | — | — |
| maintenance | | | | | | | | | | | | | | | | | | | | |
| rate | | | | | | | | | | | | | | | | | | | | |
| WSS/Kinetic | −1 | 0 | −2 | — | — | — | — | — | — | — | — | — | 1 | — | — | — | — | — | — | — |
| energy = | | | | | | | | | | | | | | | | | | | | |
| TAN\|WSS \| | | | | | | | | | | | | | | | | | | | | |
| OSI | 0 | 0 | 0 | — | — | — | — | — | — | — | — | — | — | 1 | — | — | — | — | — | — |
| High-shear | 0 | −2 | 0 | — | — | — | — | — | — | — | — | — | — | — | 1 | — | — | — | — | — |
| area/CCA | | | | | | | | | | | | | | | | | | | | |
| cross-section | | | | | | | | | | | | | | | | | | | | |
| Stenosis- | 0 | −2 | 0 | — | — | — | — | — | — | — | — | — | — | — | — | 1 | — | — | — | — |
| prone area/ | | | | | | | | | | | | | | | | | | | | |
| CCA | | | | | | | | | | | | | | | | | | | | |
| cross-section | | | | | | | | | | | | | | | | | | | | |
| Pressure loss | −1 | 0 | −2 | — | — | — | — | — | — | — | — | — | — | — | — | — | 1 | — | — | — |
| coefficient | | | | | | | | | | | | | | | | | | | | |

[symbol]

indicates data missing or illegible when filed

The dimensionless hemodynamic factors may represent physical meanings and may be utilized in the calculation of hemodynamic indices.

Regarding the carotid artery 400, it can be divided into pre-operative (Pre), post-operative (Post), and normal case (Control) that is the opposite of the occluded case.

The carotid artery 400 may create a carotid artery shape using a 3D printer from a patient's CT image, reproduce the actual pulse of the patient, and then provide velocity information.

FIG. 5 illustrates a configuration for normalizing a hemodynamic factor according to an embodiment of the present invention.

FIG. 5 illustrates a standard for calculating a hemodynamic standard factor by assigning a score to the hemodynamic factor according to an embodiment of the present invention and normalizing it.

Referring to FIG. 5, graph 500 represents PRE, which is an average value of the stenotic carotid artery immediately before surgery, CTRL, which represents the carotid artery on the opposite side of the operated carotid artery, and a threshold value 501 for distinguishing stenosis and a threshold value 502 for distinguishing hyperexpansion which are based on an average of POST that represents the expanded carotid artery immediately after surgery.

In relation to PRE, if it is above the threshold value 501 corresponding to 90%, it can be indicated as stenosis, and in relation to POST, if it is above the threshold value 502 corresponding to 10%, it can be identified as hyperexpansion.

It is assumed that the pᵢ (Π) value of each group can distinguish between stenosis and expansion according to a normal distribution.

Most PRE carotid arteries are given high scores, and very few POST carotid arteries are given low scores.

In other words, based on the pie values, shown in Table 3, and Graph 500, scores can be assigned to classify data into stenosis, normal, and hyperexpansion.

In other words, for the dimensionless hemodynamic factors, a hemodynamic standard factor ranging from 0 to 10 may be assigned using a scoring formula defined for each factor.

The score range may be flexibly changed according to the user's settings, and may be defined as in Equation 7 below in relation to the dimensionless hemodynamic factor and Equation 5.

$${HP}_{1} = \{ \begin{matrix}10 & ( {h}_{1} > 6 .. 649 {e}^{- 6} ) \\ 2.14 {e}^{6} * ( {h}_{1} - 2.07 {e}^{- 6} ) & ( 2.07 {e}^{- 6} < {h}_{1} < 6.649 {e}^{- 6} ) \\ 0 & ( {h}_{1} < 2.07 {e}^{- 6} )\end{matrix} \qquad [ Equation 7 ]$$

In Equation 7, HP₁ may represent a hemodynamic standard factor, and h₁ may represent a hemodynamic factor.

According to the scoring of the physical meaning assigned based on Equation 7, the closer to “0”, the more likely it is to be classified as a dangerous carotid artery due to severe expansion, the closer to “5”, the more likely it is to be classified as a normal carotid artery, and the closer to “10”, the more likely it is to be classified as a dangerous carotid artery due to severe stenosis.

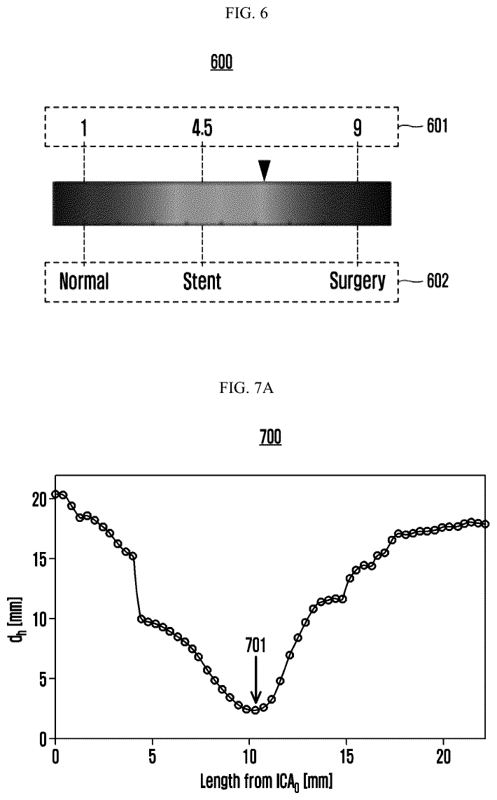

FIG. 6 is a drawing for explaining a normalized clinical guideline according to an embodiment of the present invention.

FIG. 6 illustrates the normalized clinical guideline according to an embodiment of the present invention.

Referring to FIG. 6, an image 600 shows a score 601 of an index, and a normalized clinical guideline 602.

Using the score 601 of the index, it is possible to quantitatively determine surgery and stent or other medical decision-making according to the normalized clinical guideline 602 and provide diagnostic support information to apply to the patient.

The image 600 provides a standard for medical decision-making quantitatively as the clinical guideline 602 using the smart index, the hemodynamic index, and the smart hemodynamic index.

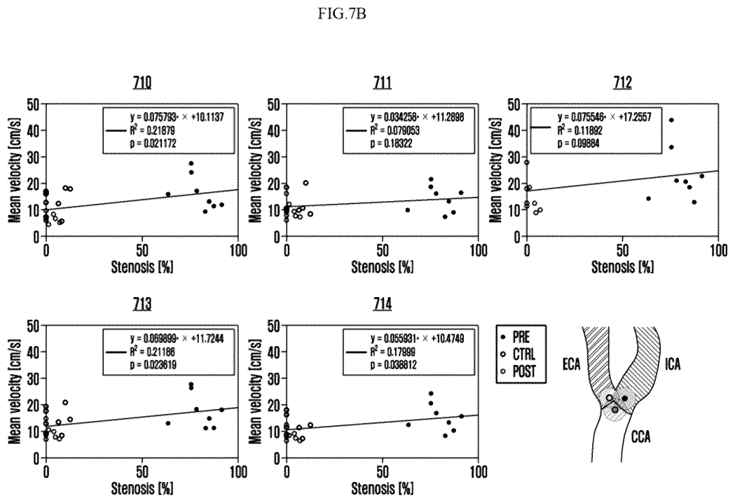

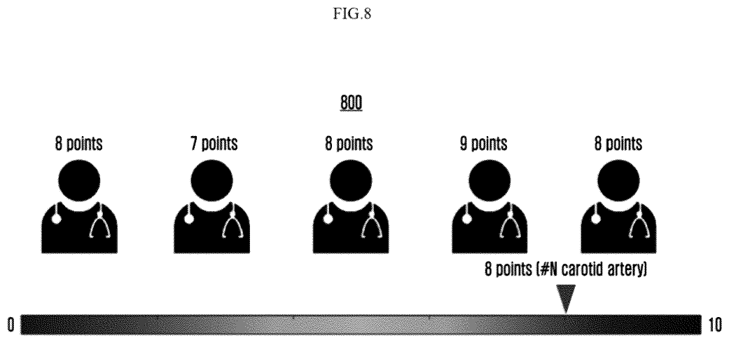

FIGS. 7a and 7b illustrate a method of calculating the weight of a hemodynamic standard factor according to an embodiment of the present invention.

FIG. 7a illustrates the degree of stenosis as a criterion for calculating the weight of a hemodynamic standard factor according to an embodiment of the present invention.

Referring to FIG. 7a, Graph 700 shows the degree of stenosis, and stenosis is identified by a part where the size of the blood vessel is maintained and a point where the blood vessel is blocked.

The degree of stenosis can be confirmed by a point 701 where the size of the blood vessel decreases and then increases or where it increases and then decreases, and since d2 is distributed linearly and the hemodynamic index and the smart hemodynamic index are indicated as merely numbers, it is important to give some meaning to the numbers so that the status can be analyzed by confirming the numbers.

The stenosis may be expressed as Equation 8 below.

$$stenosis = \frac{( {d}_{2} - ds )}{{d}_{2}} \times 100 ( \% ) \qquad [ Equation 8 ]$$

In Equation 8, d₂ may represent a length change in a point where it decreases and then increases, and dₛ may represent a peak point where it goes up and then down.

FIG. 7b illustrates the linear regression analysis results of the weight of the hemodynamic standard factor according to an embodiment of the present invention.

Referring to FIG. 7b, Graph 710 shows PRE, CTRL and POST for ICA.

Graph 711 shows PRE, CTRL and POST for CCA.

Graph 712 shows PRE, CTRL and POST for ECA.

Graph 713 shows PRE, CTRL and POST for all carotids.

Graph 714 shows PRE, CTRL and POST for a combination of ICA and CCA.

PRE may represent an average value of the stenotic carotid artery immediately before surgery, CTRL may represent the carotid artery on the opposite side of the operated carotid artery, and POST may represent the expanded carotid artery immediately after surgery.

In Graphs 710 to 714, R² represents a linearly distributed index (numerical value).

The hemodynamic index and the smart hemodynamic index may be calculated by assigning the weight (wᵢ) to the hemodynamic standard factor according to an embodiment of the present invention and performing weighted-summing.

The weight can be organized as shown in Equation 9 below for the coefficient of determination (R_squarei) of the hemodynamic standard factor by performing simple linear regression analysis on the hemodynamic standard factor and stenosis degree for a total of 24 carotid arteries. That is, the weight can be calculated using Equation 9.

$${w}_{i} = \frac{{R\_squre}_{i}}{{\sum }_{i = 1}^{n} {R\_squre}_{i}} \qquad [ Equation 9 ]$$

In Equation 9, wᵢ may represent a weight, and R_squreᵢ may represent the coefficient of determination of the weight.

In other words, the hemodynamic index may be calculated by combining the weight to the hemodynamic standard factor calculated based on the hemodynamic factor.

The smart index may be calculated in the same manner as the hemodynamic index.

FIG. 8 is a drawing for explaining a method of providing a current patient status diagnosis result according to one of a smart index, a hemodynamic index and a smart hemodynamic index according to an embodiment of the present invention.

FIG. 8 illustrates a method of providing a current patient status diagnosis result according to one of a smart index, a hemodynamic index and a smart hemodynamic index according to an embodiment of the present invention.

Referring to FIG. 8, the diagnosis AID METHOD according to an embodiment of the present invention may provide a diagnosis result according to the values of the smart index, hemodynamic index and smart hemodynamic index, and a clinical diagnostic guideline.

For example, a clinical diagnostic guideline 800 may include scores and guide information determined by multiple medical professionals based on clinical information related to a diagnosis subject.

For example, the clinical diagnostic guideline may be presented as in Table 4 below. In the following table, the clinical diagnostic guideline according to the hemodynamic index is exemplified as an index, but it may be equally applied to the smart index and the smart hemodynamic index, and some values may be changed.

TABLE 4

| Grade | Index | Clinical diagnosis guidelines |
|---|---|---|
| D₂ | 0~1 | Severe expansion, Recommendation for ICA |
| | | bulb reduction and reshaping through |
| | | reoperation/procedure |
| C₂ | 1~2 | Moderate expansion, concerns about |
| | | abnormalities due to expansion, observation |
| | | of changes is recommended |
| A | 2~5 | No abnormal findings, if the patient has a |
| | | family history or underlying disease, re- |
| | | examination is recommended after 5 years |
| | | from the examination date |
| B | 6~7 | Continuous observation, observation of |
| | | changes through re-examination within 6 |
| | | months from the examination date is recommended |
| C | 7~8 | Moderate stenosis, when there are changes |
| | | in stenosis and plaque size, active treatment |
| | | such as drug administration is recommended. |
| D | 8~9 | Severe stenosis, the degree of stenosis is |
| | | 70% or more, since the irregular shape shows |
| | | accelerated stenosis, it is recommended to |
| | | perform treatment within 3 months. |
| E | 9~10 | Fatal stenosis, high risk of cerebral |
| | | infarction, immediate treatment is recommended |

For example, the clinical diagnostic guideline 800 presents all comprehensive medical information of the patient to multiple medical professionals, and links the results of the diagnosis of the carotid artery to the index value.

Therefore, the present invention calculates the smart index and hemodynamic index, which are indices formulated using information that can be obtained from existing medical devices, and the smart hemodynamic index obtained by integrating the smart index and hemodynamic index, so the present invention does not require the purchase of new medical devices, and thus can provide a diagnosis support apparatus, which can be used in all 1st, 2nd, and 3rd medical environments, and a use method thereof.

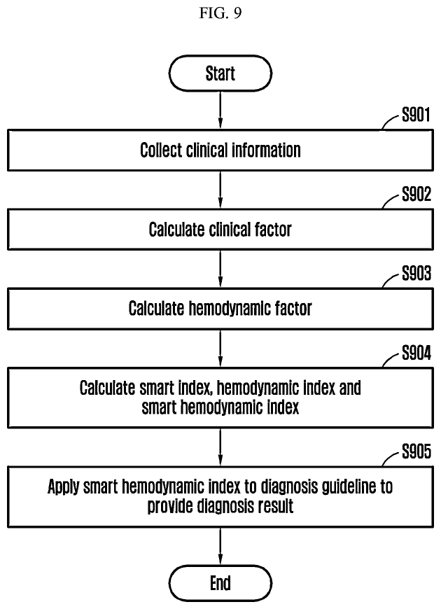

FIG. 9 is a drawing for explaining a diagnosis AID METHOD using a smart hemodynamic index according to an embodiment of the present invention.

FIG. 9 illustrates a diagnosis AID METHOD using a smart hemodynamic index according to an embodiment of the present invention.

Referring to FIG. 9, in step S901, the diagnosis AID METHOD using a smart hemodynamic index according to an embodiment of the present invention collects clinical information.

That is, the diagnosis AID METHOD using a smart hemodynamic index according to an embodiment of the present invention collects clinical information related to a diagnosis subject.

For example, a medical image including at least one of angiographic images, ultrasound Doppler images and CT images may be collected according to clinical information acquisition criteria, and data on 3D space and 4D blood flow information may be collected as clinical information by applying an artificial intelligence prediction model to the collected medical image.

In step S902, the diagnosis AID METHOD using a smart hemodynamic index according to an embodiment of the present invention calculates clinical factors.

That is, the diagnosis AID METHOD using a smart hemodynamic index according to an embodiment of the present invention may calculate clinical factors based on disease-specific characteristics in the collected clinical information.

For example, the clinical factors may be referred to as clinical parameters, and may include at least one of diabetes, blood pressure, hyperlipidemia, smoking, hematocrit, cholesterol level, pulse rate, ultrasound image, CT image, MRI image, pain, heart failure, blood concentration in the body, blood inflammation level, myocardial perfusion single photon tomography image, underlying disease, heredity, body weight and age.

In step S903, the diagnosis AID METHOD using a smart hemodynamic index according to an embodiment of the present invention calculates hemodynamic factors.

That is, the diagnosis AID METHOD using a smart hemodynamic index according to an embodiment of the present invention may calculate hemodynamic factors based on four-dimensional blood flow velocity information.

For example, the hemodynamic factor may be referred to as a hemodynamic parameter.

The hemodynamic factor may be calculated based on the dimensionless of four-dimensional blood flow velocity information in clinical information and may include at least one of basic blood flow information factors, velocity factors, flow factors, secondary flow factors, wall stress-based factors, pressure factors and morphological factors.

In step S904, the diagnosis AID METHOD using a smart hemodynamic index according to an embodiment of the present invention calculates a smart index, a hemodynamic index and a smart hemodynamic index.

That is, the diagnosis AID METHOD using a smart hemodynamic index according to an embodiment of the present invention may calculate a smart index using clinical factors, may calculate a hemodynamic index using hemodynamic factors, and may calculate a smart hemodynamic index by simultaneously considering the smart index and the hemodynamic index.

In step S905, the diagnosis AID METHOD using a smart hemodynamic index according to an embodiment of the present invention applies the smart hemodynamic index to the diagnosis guideline to provide a diagnosis result.

That is, the diagnosis AID METHOD using a smart hemodynamic index according to an embodiment of the present invention may apply one of the hemodynamic index and the smart hemodynamic index to the diagnosis guideline to provide a diagnosis result for the diagnosis subject.

Further, the diagnosis AID METHOD using a smart hemodynamic index according to an embodiment of the present invention provides the clinical information related to the diagnosis subject to multiple medical professionals, and then applies a score, based on one of the indices, to a diagnostic guideline determined based on the diagnosis result, thereby providing a diagnostic result corresponding to the score applied in the diagnostic guideline.

Therefore, the present invention provides a hemodynamic index and a smart index, which are innovative diagnostic technologies capable of expressing the analysis process and results, which require a high level of understanding of both hemodynamics and medicine, as quantitatively normalized numbers and capable of suggesting a clinical guideline that matches the numbers, and a smart hemodynamic index obtained by integrating the hemodynamic index and the smart index, thereby innovatively simplifying an existing medical diagnosis process and, at the same time, supporting accurate diagnosis and prognosis prediction through more multifaceted considerations.

The apparatus described above may be implemented as a hardware component, a software component, and/or a combination of hardware components and software components. For example, the apparatus and components described in the embodiments may be achieved using one or more general purpose or special purpose computers, such as, for example, a processor, a controller, an arithmetic logic unit (ALU), a digital signal processor, a microcomputer, a field programmable array (FPA), a programmable logic unit (PLU), a microprocessor, or any other device capable of executing and responding to instructions. The processing device may execute an operating system (OS) and one or more software applications executing on the operating system. In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

Although the present invention has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

1. A diagnosis AID DEVICE using a smart hemodynamic index, the diagnosis AID DEVICE comprising:
a clinical information collector for collecting clinical information related to a diagnosis subject;
a clinical factor calculator for calculating a clinical factor based on the collected clinical information;
a hemodynamic factor calculator for calculating a hemodynamic factor based on the collected clinical information;
an index calculator for calculating a smart index (SI) using the calculated clinical factor, calculating a hemodynamic index (HDI) using the calculated hemodynamic factor, and calculating a smart hemodynamic index (SHDI) by simultaneously considering the calculated smart index and the calculated hemodynamic index; and
a diagnosis result provider for providing a diagnosis result for the diagnosis subject by applying one of the calculated smart index, the calculated hemodynamic index and the calculated smart hemodynamic index to a diagnosis guideline.

2. The diagnosis AID DEVICE according to claim 1, wherein the index calculator calculates a hemodynamic standard factor by normalizing the hemodynamic factor, and calculates the hemodynamic index by assigning a weight to the calculated hemodynamic standard factor.

3. The diagnosis AID DEVICE according to claim 1, wherein the index calculator calculates a clinical standard factor by normalizing the clinical factor, and calculates the smart index by assigning a weight to the calculated clinical standard factor.

4. The diagnosis AID DEVICE according to claim 1, wherein the clinical factor calculator calculates a clinical factor comprising at least one of diabetes, blood pressure, hyperlipidemia, smoking, hematocrit, cholesterol level, pulse rate, ultrasound image, CT image, MRI image, pain, heart failure, blood concentration in the body, blood inflammation level, myocardial perfusion single photon tomography image, underlying disease, heredity, body weight and age based on disease-specific characteristics in the clinical information.

5. The diagnosis AID DEVICE according to claim 1, wherein the diagnosis result provider provides clinical information, related to the diagnosis subject, to multiple medical professionals, and then, by applying a score, based on one of the indices, to a diagnosis guideline determined based on a diagnosis result, provides a diagnosis result corresponding to the applied score in the diagnosis guideline.

6. The diagnosis AID DEVICE according to claim 1, wherein the clinical information collector collects a medical image comprising at least one of angiographic images, ultrasound Doppler images and CT images according to clinical information acquisition criteria, and collects data on 3D space and 4D blood flow information as clinical information by applying an artificial intelligence prediction model to the collected medical image.

7. The diagnosis AID DEVICE according to claim 1, wherein the hemodynamic factor calculator calculates a hemodynamic factor comprising at least one of basic blood flow information factors, velocity factors, flow factors, secondary flow factors, wall stress-based factors, pressure factors and morphological factors based on four-dimensional blood flow velocity information in the clinical information.

8. The diagnosis AID DEVICE according to claim 7, wherein the index calculator calculates a hemodynamic standard factor by assigning a score in a predefined score range according to the calculated hemodynamic factor, classifies a disease into one among a plurality of diseases according to the calculated hemodynamic standard factor, and calculates a hemodynamic index by assigning a weight, calculated according to a degree of disease, to the calculated hemodynamic standard factor.

9. The diagnosis AID DEVICE according to claim 8, wherein the index calculator classifies the calculated hemodynamic factor into multiple cases, calculates a threshold value for each disease based on an average value of each of the classified case, and calculates a hemodynamic standard factor by assigning a score in the predefined score range based on the calculated threshold value.

10. The diagnosis AID DEVICE according to claim 1, wherein the index calculator calculates a clinical standard factor by assigning a score in a predefined score range according to the calculated clinical factor, classifies a disease into one of a plurality of diseases according to the calculated clinical standard factor, and calculates a smart index by assigning a weight, calculated according to a degree of disease, to the calculated clinical standard factor.

11. A diagnosis AID METHOD using a smart hemodynamic index, the diagnosis AID METHOD comprising:
collecting, by a clinical information collector, clinical information related to a diagnosis subject;
calculating, by a clinical factor calculator, a clinical factor based on the collected clinical information;
calculating, by a hemodynamic factor calculator, a hemodynamic factor based on the collected clinical information;
calculating, by an index calculator, a smart index (SI) using the calculated clinical factor, calculating a hemodynamic index (HDI) using the calculated hemodynamic factor, and calculating a smart hemodynamic index (SHDI) configured by considering the calculated smart index and the calculated hemodynamic index; and
providing, by a diagnosis result provider, a diagnosis result for the diagnosis subject by applying one of the calculated smart index, the calculated hemodynamic index and the calculated smart hemodynamic index to a diagnosis guideline.

12. The diagnosis AID METHOD according to claim 11, wherein the calculating by the index calculator comprises:
calculating a hemodynamic standard factor by normalizing the hemodynamic factor, and calculating the hemodynamic index by assigning a weight to the calculated hemodynamic standard factor,
calculating a clinical standard factor by normalizing the clinical factor, and calculating the smart index by assigning a weight to the calculated clinical standard factor; and
calculating a clinical standard factor by normalizing the clinical factor, and calculating the smart index by assigning a weight to the calculated clinical standard factor.

13. The diagnosis AID METHOD according to claim 11, wherein the providing by the diagnosis result provider comprises:
providing clinical information, related to the diagnosis subject, to multiple medical professionals, and then, by applying a score, based on one of the indices, to a diagnosis guideline determined based on a diagnosis result, providing a diagnosis result corresponding to the applied score in the diagnosis guideline.

14. The diagnosis AID METHOD according to claim 11, wherein the collecting by the clinical information collector comprises:
collecting a medical image comprising at least one of angiographic images, ultrasound Doppler images and CT images according to clinical information acquisition criteria, and collecting data on 3D space and 4D blood flow information as clinical information by applying an artificial intelligence prediction model to the collected medical image.

15. The diagnosis AID METHOD according to claim 11, wherein the calculating by the hemodynamic factor calculator comprises:
calculating a hemodynamic factor comprising at least one of basic blood flow information factors, velocity factors, flow factors, secondary flow factors, wall stress-based factors, pressure factors and morphological factors based on four-dimensional blood flow velocity information in the clinical information,
the calculating by the index calculator comprises:
calculating a hemodynamic standard factor by assigning a score in a predefined score range according to the calculated hemodynamic factor, classifying a disease into one among a plurality of diseases according to the calculated hemodynamic standard factor, and calculating a hemodynamic index by assigning a weight, calculated according to a degree of disease, to the calculated hemodynamic standard factor.

16. The diagnosis AID METHOD according to claim 11, wherein the calculating by the clinical factor calculator comprises:
calculating a clinical factor comprising at least one of diabetes, blood pressure, hyperlipidemia, smoking, hematocrit, cholesterol level, pulse rate, ultrasound image, CT image, MRI image, pain, heart failure, blood concentration in the body, blood inflammation level, myocardial perfusion single photon tomography image, underlying disease, heredity, body weight and age based on disease-specific characteristics in the clinical information,
the calculating by the index calculator comprises:
calculating a clinical standard factor by assigning a score in a predefined score range according to the calculated clinical factor, classifying a disease into one of a plurality of diseases according to the calculated clinical standard factor, and calculating a smart index by assigning a weight, calculated according to a degree of disease, to the calculated clinical standard factor.
